(19) United States
(12) Patent Application Publication
(10) Pub. No.: US 2013/0280720 A1
(43) Pub. Date: Oct. 24, 2013

(54) TISSUE BIOMARKERS FOR INDICATION OF PROGRESSION FROM BARRETT'S ESOPHAGUS TO ESOPHAGEAL ADENOCARCINOMA

(71) Applicant: THE UNIVERSITY OF KANSAS, Lawrence, KS (US)

(72) Inventors: Lane Kevin Christenson, Kansas City, KS (US); Ajay Bansal, Mission Hills, KS (US); Prateek Sharma, Leawood, KS (US)

(21) Appl. No.: 13/798,932

(22) Filed: Mar. 13, 2013

Related U.S. Application Data
(60) Provisional application No. 61/685,388, filed on Mar. 15, 2012.

(51) Int. Cl. C12Q 1/68

(52) U.S. Cl. CPC C12Q 1/6886
USPC 435/6.12

(57) ABSTRACT

A method of diagnosing progression from Barrett's esophagus toward esophageal dysplasia can include: obtaining miRNA from a test subject having Barrett's esophagus; assaying for miRNA biomarker selected from one or more of miR-15b or miR-486-5p; and determining whether one or more of miR-15b or miR-486-5p provide an indication of progression toward esophageal dysplasia in the test subject. The method can include determining an amount of one or more of miR-15b or miR-486-5p. The method can include determining a modulation in amount of one or more of miR-15b or miR-486-5p. The method can include comparing the one or more of miR-15b or miR-486-5p with a positive control or a negative control.

### CROSS-REFERENCE TO RELATED APPLICATIONS

This patent application claims priority to U.S. Provisional Patent Application Ser. No. 61/685,388 filed Mar. 15, 2012, which is incorporated herein by specific reference in its entirety.

### SEQUENCE LISTING

The instant application contains a Sequence Listing which has been submitted in ASCII format via EFS-Web and is hereby incorporated by reference in its entirety. Said ASCII copy, created on Mar. 12, 2013, is named K1262.10032US01_SL.txt and is 2,047 bytes in size.

### BACKGROUND

Esophageal adenocarcinoma (EAC) can be a serious health complication. It has been found that the presence of Barrett's esophagus (BE) in a subject increases the risk of EAC by up to 30-40 fold. Currently, histologic identification of dysplasia is used to identify subjects that have BE patients that have a high risk for progression to adenocarcinoma. However, disease progression is thought to begin with BE and evolve through the stages of non-dysplastic intestinal metaplasia, low grade dysplasia (LGD), high grade dysplasia (HGD) and finally cancer, such as EAC. However, histologic identification of dysplasia may not be practical in some instances, or it may be too late and the progression to cancer may already be substantial. Therefore, it is advantageous to identify new procedures for predicting or diagnosing susceptibility to LGD or HGD or early stage progression toward LGD or HGD. Also, it is advantageous to identify new procedures for diagnosing susceptibility to or progression toward esophageal adenocarcinoma.

### SUMMARY

In one embodiment, a method of diagnosing progression from Barrett's esophagus toward esophageal dysplasia/cancer can include: obtaining miRNA from a test subject having Barrett's esophagus; assaying for miRNA selected from one or more of miR-15b or miR-486-5p; and determining whether one or more of miR-15b or miR-486-5p provide an indication of progression toward esophageal dysplasia/cancer in the test subject. The method can include determining an amount of one or more of miR-15b or miR-486-5p. The method can include determining a modulation in amount of one or more of miR-15b or miR-486-5p. The method can include comparing one or more of miR-15b or miR-486-5p with a positive control or a negative control, the positive control being from a subject having esophageal dysplasia/cancer, the negative control being from a subject without esophageal dysplasia. In one aspect, if one or more of the miR-15b or miR-486-5p is similar to the positive control or different from the negative control, there is an indication of esophageal dysplasia/cancer in the test subject. In one aspect, if one or more of the miR-15b or miR-486-5p is similar to the negative control or different from the positive control, there is an indication of esophageal dysplasia in the test subject.

In one embodiment, the method of diagnosing progression from Barrett's esophagus toward esophageal dysplasia can include assaying for miRNA selected from one or more of miR-21, miR-145, miR-203, or miR-let-7a; and determining whether one or more of miR-21, miR-145, miR-203, or miR-let-7a provide an indication of esophageal dysplasia/cancer in the test subject.

In one embodiment, the assaying can include contacting the miRNA with a complement thereof. In one aspect, the complement being located on a diagnostic chip.

In one embodiment, the method can include performing RT-PCR with one or more primers for the miRNA.

In one embodiment, a method of detecting progression from Barrett's esophagus toward dysplasia/cancer can include: obtaining miRNA from a test subject; assaying for a plurality of miRNA selected from the group consisting of miR-15b, miR-486-5p, miR-21, miR-145, miR-203, or miR-let-7a; and determining whether the assayed plurality of miRNA selected from the group consisting of miR-15b, miR-486-5p, miR-21, miR-145, miR-203, or miR-let-7a provide an indication of progression toward dysplasia in the test subject. The method can include determining an amount or modulation of the assayed plurality of miRNA selected from the group consisting of miR-15b, miR-486-5p, miR-21, miR-145, miR-203, or miR-let-7a. The method can include comparing the assayed plurality of miRNA selected from the group consisting of miR-15b, miR-486-5p, miR-21, miR-145, miR-203, or miR-let-7a with a positive control or a negative control, the positive control being from a subject having dysplasia, the negative control being from a patient without dysplasia. If one or more of the miR-15b or miR-486-5p is similar to the positive control or different from the negative control, there is an indication of progression toward dysplasia/cancer in the test subject. If one or more of the miR-15b or miR-486-5p is similar to the negative control or different from the positive control, there is an indication of progression toward dysplasia/cancer in the test subject.

In one aspect, the methods can include diagnosing esophageal dysplasia in the test subject. In one aspect, the methods can include diagnosing esophageal cancer in the test subject.

In one embodiment, a diagnostic kit can include: a plurality of polynucleotides selected from the group consisting of miR-15b, miR-486-5p, miR-21, miR-145, miR-203, miR-let-7a, or compliment thereof. The kit can include one or more of: miR-15b or complement thereof; or miR-486-5p or complement thereof. In one aspect, the plurality of nucleotides can include one or more primers for two or more of miR-15b, miR-486-5p, miR-21, miR-145, miR-203, or miR-let-7a. In one aspect, the plurality of nucleotides can include a compliment polynucleotide for two or more of miR-15b, miR-486-5p, miR-21, miR-145, miR-203, or miR-let-7a.

In one embodiment, a biomarker chip can include a plurality compliment nucleotides for two or more of miR-15b, miR-486-5p, miR-21, miR-145, miR-203, or miR-let-7a.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 includes diagrams that illustrate biopsy schema. The left panel represents Group A, which includes intestinal metaplasia tissues from BE patients without dysplasia. The right panel illustrates non-dysplastic tissues (Group B) and dysplastic tissues (Group C) from BE patients with high-grade dysplasia/early esophageal cancer.

FIG. 2 includes graphs of data that illustrate quantitative RT-PCR validation of differential expression of miRNA (miR-15b, miR-21, miR-145, miR-203, miR-486-5p, and miR-let-7a) in BE patients across the three tissue groups: Group A; Group B; and Group C. The results are depicted as fold change following normalization with small nucleolar RNAU6. The tissue includes intestinal metaplasia (i.e., IM) and high-grade dysplasia/esophageal adenocarcinoma (i.e., HGD). BE represents tissue from BE patients with non-dysplastic intestinal metaplasia. BE-HGD represents tissue from BE patients with high-grade dysplasia/esophageal adenocarcinoma. The “a” and/or “b” above the graph bars indicate means±s.e.m with different superscript are different P<0.05.

FIG. 3 includes graphs of data that illustrate performance characteristics of miRNA (miR-15b, miR-21, miR-203, miR-486-5p, and miR-let-7a) for the diagnosis of dysplasia/cancer (e.g., Groups A vs. C), where fold change with “+” prefix indicates up regulation and “−” prefix indicates down regulation and the best performing cutoff.

FIG. 4 includes graphs that illustrate performance characteristics of miRNA (miR-15b, miR-21, miR-203, miR-486-5p, and miR-let-7a) for the diagnosis of dysplasia/cancer (e.g., Groups A vs. B+C), where fold change with “+” prefix indicates up regulation and “−” prefix indicates down regulation and the best performing cutoff.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention includes systems and method for using select miRNA as biomarkers in determining whether a patient is susceptible to or has already undergone progression from BE to HGD/EAC. The select miRNA biomarkers can be obtained from tissue or serum of BE patients, which have been diagnosed or have BE. The miRNA biomarkers can be obtained and assayed and compared to standards or controls as described herein. The miRNA biomarkers can be obtained and assayed in order to determine sensitivity and selectivity for indicating dysplasia, which provide indications of susceptibility to or undergoing the progression from BE to LGD and/or HGD and/or EAC. Once susceptibility to or undergoing the progression from BE to LGD and/or HGD and/or EAC has been identified through miRNA biomarker analysis, therapeutic intervention can be undertaken to inhibit or reduce the progression. Also, the miRNA biomarkers can be assayed to determine whether or not to LGD and/or HGD and/or EAC has already developed, which detection can result in therapeutic treatment.

In one embodiment, the present invention can also include systems and methods for determining additional miRNA biomarkers that can be used in the diagnostic assays for indications of susceptibility to or undergoing the progression from BE to LGD and/or HGD and/or EAC. The methods can be employed with other miRNA to determine whether the miRNA are differentially expressed in BE patients with and without LGD and/or HGD and/or EAC. When the miRNA are differentially expressed in tissues having LGD and/or HGD and/or EAC compared to non-dysplasia BE tissue, the miRNA can be biomarkers for predicting or diagnosing progression to LGD and/or HGD and/or EAC.

In one embodiment, the present invention can include systems and methods for determining whether tissue miRNAs biomarkers are differentially expressed in BE patients with HGD/EAC and without HGD/EAC. The systems and methods can be used to determine whether miRNA expression in non-dysplastic tissues from patients with HGD have similar or different profiles compared to non-dysplastic tissues from patient without HGD. The miRNA having different expression in patients without HGD and patients with HGD can then be used as biomarkers for diagnostics with BE patients to determine whether the BE patients are susceptible or undergoing progression toward HGD. The differentially expressed miRNA between patients without HGD and patients with HGD can be biomarkers for progression from BE toward LGD and/or HGD and/or EAC. The expression profiles of the selected miRNA biomarkers can then be used in prediction of or diagnosis of susceptibility or progression from BE toward LGD and/or HGD and/or EAC.

In one embodiment, the diagnostic systems and method can examine paired non-dysplastic and dysplastic tissues from the same patients. Optionally, the diagnostics can be performed with reference to the amount of primary lesion within tissue biopsy. In one embodiment, the diagnostic systems and method can examine non-dysplastic and dysplastic tissues from different patients. Optionally, the diagnostics can be performed with reference to the amount of primary lesion within tissue biopsy.

In one embodiment, performance of the diagnostics can include analysis of histologic details of biopsy specimens having a lesion of interest (i.e. metaplasia and/or dysplasia), and selection of specimens for miRNA analysis when the lesion occupied greater than 50% of the specimen surface area. However, the lesion may occupy less than 50%, such as about 40%, about 30%, about 25%, or even less as long as miRNA can be obtained. The diagnostics of the present invention can have increased likelihood of detecting real differences in miRNA expression by only including those biopsies where the lesion of interest (i.e. metaplasia and/or dysplasia) is present. Accordingly, the diagnostics can include obtaining a tissue specimen, and confirming the lesion of interest is present in the tissue specimen before obtaining miRNA from the tissue specimen.

In one embodiment, performance of the diagnostics can include obtaining miRNA from serum. The serum miRNA can be analyzed to determine if there is a change in expression compared to controls or standards. The controls can include positive controls from HGD/EAC positive subjects or tissues thereof and negative controls from HGD/EAC negative subjects or tissues thereof. Serum can be obtained by standard procedures.

In one embodiment, the miRNA identified and described in this application as biomarkers for progression toward HGD/EAC can be used in the diagnostics for prediction of susceptibility or progression toward HGD/EAC. The miRNA used in the diagnostics can have reasonable clinical accuracy, such as sensitivity of about 87% to about 93% and/or specificity of about 55% to about 90% for prediction of susceptibility to or progression to HGD/EAC.

In one embodiment, the miRNA described herein can be considered biomarkers for susceptibility or development of the neoplasia. In one aspect, the miRNA described herein can be used to diagnose presence of neoplasia.

In one embodiment, one or more of the miRNA identified in the unbiased high-throughput screen described herein can be used in the diagnostics. The identified miRNA associated with dysplasia in BE patients that have modulated expression between non-dysplasia and dysplasia positive subjects. These miRNA can be used as biomarkers and assayed in test subjects to determine a change compared to a standard or control that has statistical significance (e.g., using qRT-PCR analysis) between BE (group A) and BE-HGD/EAC (Group C) samples (e.g., tissues or serum).

In one embodiment, the diagnostics can assay for modulation of miRNA expression that can provide an early indication of susceptibility of or early progression toward neoplastic state. The early diagnostic can use miR-21, which has been found to have a pronounced increase of about 6-fold increase in Group B tissues compared to Group A tissues. It has been postulated, without being bound thereto, that the increase in miR-21 expression provides an indication of susceptibility or early stage progression toward neoplastic state. Accordingly, miR-21 can be used as an early marker for progression toward HGD/EAC. As such, miR-21 or complement thereof can be included in an assay kit or diagnostic procedure with or without other miRNA described herein. However in an alternate embodiment, the present inventive kits or diagnostic processes may exclude miR-21 or complement thereof. A complement polynucleotide can include complete complementarity, or at least 99%, or at least 95%, or at least 90%, or at least 85%, or at least 80%, or at least 75% complementarity between polynucleotides.

In one embodiment, the diagnostics can assay for modulation of miR-let-7a to provide an indication of susceptibility or progression toward neoplastic state. As described herein, microarray results indicated that miR-let-7a can be down regulated or reduced in expression in Group C tissues versus Group A tissues. The down regulation of miR-let-7a can be used in diagnostics to provide an indication of progression toward HGD/EAC. As such, miR-let-7a or complement thereof can be included in an assay kit or diagnostic procedure with or without other miRNA described herein. However in an alternate embodiment, the present inventive kits or diagnostic processes may exclude miR-let-7a or complement thereof.

In one embodiment, the diagnostics can assay for modulation of miR-205 to provide an indication of susceptibility or progression toward neoplastic state, when used with the other miRNA described herein. The analysis of miR-205 can provide an indication that is supported by an indication of another miRNA. In one aspect, analysis of miR-205 may not be performed alone for an indication, but modulation of miR-205 consistent with modulation of one of the other miRNA specifically described herein can indeed provide an indication. However in an alternate embodiment, the present inventive kits or diagnostic processes may exclude miR-205 or complement thereof.

In one embodiment, the diagnostics can assay for modulation of miR-145 to provide an indication of susceptibility or progression toward neoplastic state, when used with the other miRNA described herein. As a biomarker, miR-145 exhibited marked (>2.9-fold) differences in expression between Group A and Group C tissues. The analysis of miR-145 can provide an indication that is supported by an indication of another miRNA. In one aspect, analysis of miR-145 may not be performed alone for an indication, but modulation of miR-145 consistent with modulation of one of the other miRNA specifically described herein can indeed provide an indication. However in an alternate embodiment, the present inventive kits or diagnostic processes may exclude miR-145 or complement thereof.

In one embodiment, the diagnostics can assay for modulation of miR-196a to provide an indication of susceptibility or progression toward neoplastic state, when used with the other miRNA described herein. As a biomarker, miR-196a exhibited marked (>2.9-fold) differences in expression between Group A and Group C tissues. The analysis of miR-196a can provide an indication that is supported by an indication of another miRNA. In one aspect, analysis of miR-196a may not be performed alone for an indication, but modulation of miR-196a consistent with modulation of one of the other miRNA specifically described herein can indeed provide an indication. However in an alternate embodiment, the present inventive kits or diagnostic processes may exclude miR-196a or complement thereof.

In one embodiment, the diagnostics can assay for modulation of miR-375 to provide an indication of susceptibility or progression toward neoplastic state, when used with the other miRNA described herein. As a biomarker, miR-375 has been found in a larger cohort of patients by qRT-PCR analysis. The analysis of miR-375 can provide an indication that is supported by an indication of another miRNA. In one aspect, analysis of miR-375 may not be performed alone for an indication, but modulation of miR-375 consistent with modulation of one of the other miRNA specifically described herein can indeed provide an indication. However in an alternate embodiment, the present inventive kits or diagnostic processes may exclude miR-375 or complement thereof.

In one embodiment, the diagnostics can assay for modulation of miRNA expression for miR-203 that can provide an early indication of susceptibility of or early progression toward neoplastic state. The early diagnostic can use miR-203, which has been found to have a pronounced increase of greater than 2.9-fold or about 11-fold increase in Group C tissues compared to Group A tissues. Also, miR-203 exhibited a 5.7-fold difference (P<0.5) by qRT-PCR in independent patients. It has been postulated, without being bound thereto, that the increase in miR-203 expression provides an indication of susceptibility or early stage progression toward neoplastic state. Accordingly, miR-203 can be used as an early marker for HGD/EAC. As such, miR-203 or complement thereof can be included in an assay kit with or without other miRNA described herein. However in an alternate embodiment, the present inventive kits or diagnostic processes may exclude miR-203 or complement thereof.

In one embodiment, the diagnostics can assay for modulation of miRNA expression for miR-15b that can provide an early indication of susceptibility of or early progression toward neoplastic state. The early diagnostic can use miR-15b, which has been found to have a pronounced increase in Group B tissues and Group C tissues compared to group A tissues. It has been postulated, without being bound thereto, that the increase in miR-15b expression provides an indication of susceptibility or early stage progression toward neoplastic state. Accordingly, miR-15b can be used as an early marker for HGD/EAC. As such, miR-15b or complement thereof can be included in an assay kit or diagnostic procedure with or without other miRNA described herein.

In one embodiment, the diagnostics can assay for modulation of miRNA expression for miR-486-5p that can provide an early indication of susceptibility of or early progression toward neoplastic state. The early diagnostic can use miR-486-5p, which has been found to have a pronounced decrease or down regulation in HGD patients compared to Group A patients. It has been postulated, without being bound thereto, that the decrease in miR-486-5p expression provides an indication of susceptibility or early stage progression toward neoplastic state. Accordingly, miR-486-5p can be used as an early marker for HGD/EAC. As such, miR-486-5p or complement thereof can be included in an assay kit or diagnostic procedure with or without other miRNA described herein.

In one embodiment, a diagnostic kit or procedure can include a combination of miR-15b and miR-486-5p. While one embodiment may also include other miRNA described herein, a specific embodiment includes only miR-15b and miR-486-5p as biomarkers. Other miRNA, such as those not described to be modulated between Group A tissues and Group B and/or Group C may be present as controls in a kit or procedure. In one aspect, a diagnostic kit or procedure can specifically exclude one or more of miR-21, miR-203, and miR-let-7A or complement thereof. In one aspect, a diagnostic kit or procedure can specifically exclude one or more of miR-21, miR-203, miR-let-7A, miR-205, miR-192, miR-196a, miR-375, miR-584, miR-1246, miR-let-7d or complement thereof.

In one embodiment, the present invention can include a diagnostic method with a process for determining whether or not miRNA modulation data is subject to field effect. For example, the data provided herein indicates that some miRNA dis-regulation in BE patients associated dysplasia may be subject to field effect. The process for determining whether or not miRNA modulation data is subject to field effect can avoid using paired non-dysplastic and dysplastic BE tissues from the same patients. The process for determining whether or not miRNA modulation data is subject to field effect can include assaying miRNA from tissues of independent BE patients with and without dysplasia, which can be used as controls to compare against a subject in need or undergoing the diagnostic procedure. In one aspect, the control miRNA can be obtained from tissues selected from a prospective tissue repository. In one aspect, the test or control miRNA can be evaluated for field effects in BE associated miRNA expression. For example, comparison of miR-15b, miR-203 and miR-21 expression in non-dysplastic tissue from patients with HGD/EAC (e.g., Group B biopsy) and without HGD/EAC (e.g., Group A biopsy) indicated that HGD within the Group B patients altered the miRNA expression of the non-dysplastic tissues. These data support the presence of an esophagus wide field effect for abnormal molecular expression in BE patients with HGD/EAC. Field effect has important clinical implications. In one example, the miRNA expression profile results may stratify BE patient into high or low risk for HGD/EAC independent of the endoscopic sampling location. As such, ablation of BE can be applied on the entire length of BE, and not limited to only dysplastic areas.

In one embodiment, the diagnostic kit or procedures of the present invention can have miRNA with high sensitivity and/or high specificity for differentiating BE patients with and without dysplasia. In one aspect, the miRNA have both high sensitivity and high specificity for differentiating BE patients with and without dysplasia.

The miRNA can have high sensitivity, which can be at least about 80%, preferably, at least about 85%, more preferably, at least about 90%, more preferably at least about 95%, more preferably at least about 98%, and most preferably at least about 99%. For example, the miRNA miR-15b, miR-21, miR-203, miR-486-5p and miR-let-7a have shown sensitivity of from about 87-93%. However, it is expected that diagnostic protocols can be enhanced to improve sensitivity. Also, the protocols may be performed to show slightly lower sensitivity, but which when combined with specificity can provide an indication of whether or not the subject is susceptible to or is progressing to HGD/EAC.

The miRNA can have high specificity, which can be at least about 50%, preferably, at least about 60%, more preferably, at least about 70%, more preferably at least about 80%, more preferably at least about 95%, and most preferably at least about 99%. For example, the miRNA miR-15b, miR-21, miR-203, miR-486-5p and miR-let-7a have shown specificity of from about 55-90%. However, it is expected that diagnostic protocols can be enhanced to improve specificity. Also, the protocols may be performed to show slightly lower specificity, but which when combined with sensitivity can provide an indication of whether or not the subject is susceptible to or is progressing to HGD/EAC.

In one embodiment, the miRNA of the invention can be obtained from a sample, such as a tissue sample or serum, from a patient undergoing the diagnostic procedure. In one aspect, the tissue can be from the gastrointestinal tract. In another aspect, the tissue can be from the esophagus. The tissue can be obtained by standard dissection or micro-dissection techniques. In one example, the micro-dissection can be by laser capture micro-dissection.

In one embodiment, the diagnostic kits and procedures can be used to predict risk of BE progressing toward HGD/EAC, which can be by identification of modulation of expression of select miRNA biomarkers described herein. Also, the diagnostic kits and procedures can be used to determine stratification BE progressing to HGD/EAC, were amount of modulation of expression of select miRNA can be used to determine if the dysplasia is low to high grade. Low modulation may indicate low stratification, while high modulation may indicate high stratification. However, the miRNA biomarkers described herein can predict progression from BE with no dysplasia to BE having HGD/EAC.

In one embodiment, the miRNA can be used for detecting or diagnosing progression from BE without dysplasia to dysplasia in BE, and progression to onset of esophageal cancer, such as EAC. However, the miRNA may also be used for detecting or diagnosing dysplasia in BE, where the patients being diagnosed may or may not have been previously diagnosed with BE. Similarly, the miRNA may be used for detecting or diagnosing esophageal cancer, such as EAC, in a patient that may or may not have been previously diagnosed with BE. The miRNA may be used for detecting or diagnosing BE in patients that have not previously been diagnosed with BE; however, such diagnosis also diagnoses dysplasia and/or esophageal cancer.

In one embodiment, a method for detecting BE can include: collecting an esophageal tissue sample; and screening the esophageal tissue sample for expression of or change in expression of miRNA selected from the group consisting of miR-15b and miR-486-5p. Such detection of expression can be modulated expression from a standard or control. For example, the standard or control can include comparison to levels of miR-15b and miR-486-5p in subjects not having BE or having BE without dysplasia. That is, the miRNA are compared to like miRNA. The screening may include: determining an expression level of one or more of miR-15b and miR-486-5p in the esophageal tissue sample, and comparing the expression level of one or more of miR-15b and miR-486-5p in the esophageal tissue sample to an expression level of one or more of miR-15b and miR-486-5p in a tissue sample not associated with BE. Also, the screening may include: providing one or more primers configured to anneal to one or more of miR-15b and miR-486-5p; amplifying one or more of miR-15b and miR-486-5p, if present in the esophageal tissue sample, using a polymerase; and comparing an amount of one or more of miR-15b and miR-486-5p in the esophageal tissue sample to an amount of one or more of miR-15b and miR-486-5p in a tissue sample not having BE or having BE without dysplasia. The primers can be 5′ primers and/or 3′ primers. The miRNA biomarkers can be identified using biochips with complementary polynucleotides or by using standard PCR techniques, such as RT-PCR as described herein. The kits and diagnostic procedures can use the biochips and/or PCR primers and reagents. This method may also be modified in that the detection of BE also detects the presence of BE dysplasia and/or esophageal cancer.

In one embodiment, a method for detecting dysplasia in BE patients can include: collecting an esophageal tissue sample; and screening the esophageal tissue sample for expression of miRNA selected from the group consisting of miR-15b and miR-486-5p. Such detection of expression can be modulated expression from a standard or control. For example, the standard or control can include comparison to levels of miR-15b and miR-486-5p in subjects not having BE or having BE without dysplasia or esophageal cancer. That is, the miRNA are compared to like miRNA. The screening may include: determining an expression level of one or more of miR-15b and miR-486-5p in the esophageal tissue sample, and comparing the expression level of one or more of miR-15b and miR-486-5p in the esophageal tissue sample to an expression level of one or more of miR-15b and miR-486-5p in a tissue sample not having BE or having BE without dysplasia or esophageal cancer. A change in expression level compared to samples not having BE or having BE without dysplasia provides an indication that the sample can include dysplasia or esophageal cancer. Also, the screening may include: providing one or more primers configured to anneal to one or more of miR-15b and miR-486-5p; amplifying one or more of miR-15b and miR-486-5p, if present in the esophageal tissue sample, using a polymerase; and comparing an amount of one or more of miR-15b and miR-486-5p in the esophageal tissue sample to an amount of one or more of miR-15b and miR-486-5p in a tissue sample not having BE or having BE without dysplasia or esophageal cancer. The primers can be 5′ primers and/or 3′ primers. Here, modulation of amount of miRNA provides an indication the sample has dysplasia or esophageal cancer.

In one embodiment, a method for detecting esophageal cancer, such as EAC, in BE patients can include: collecting an esophageal tissue sample; and screening the esophageal tissue sample for expression of miRNA selected from the group consisting of miR-15b and miR-486-5p. Such detection of expression can be modulated expression from a standard or control. For example, the standard or control can include comparison to levels of miR-15b and miR-486-5p in subjects not having BE or having BE without dysplasia or esophageal cancer. That is, the miRNA are compared to like miRNA. The screening may include: determining an expression level of one or more of miR-15b and miR-486-5p in the esophageal tissue sample, and comparing the expression level of one or more of miR-15b and miR-486-5p in the esophageal tissue sample to an expression level of one or more of miR-15b and miR-486-5p in a tissue sample not having BE or having BE without dysplasia or esophageal cancer. A change in expression level compared to samples not having BE or having BE without dysplasia provides an indication that the sample can include esophageal cancer. Also, the screening may include: providing one or more primers configured to anneal to one or more of miR-15b and miR-486-5p; amplifying one or more of miR-15b and miR-486-5p, if present in the esophageal tissue sample, using a polymerase; and comparing an amount of one or more of miR-15b and miR-486-5p in the esophageal tissue sample to an amount of one or more of miR-15b and miR-486-5p in a tissue sample not having BE or having BE without dysplasia or esophageal cancer. The primers can be 5′ primers and/or 3′ primers. Here, modulation of amount of miRNA provides an indication the sample has esophageal cancer.

In one embodiment, a method for detecting BE can include: collecting an esophageal tissue sample; and screening the esophageal tissue sample for expression of or change in expression of miRNA selected from miR-15b, miR-21, miR-203, miR-486-5p, and miR-let-7a. Such detection of expression can be modulated expression from a standard or control. For example, the standard or control can include comparison to levels of miRNA selected from miR-15b, miR-21, miR-203, miR-486-5p, and miR-let-7a in subjects not having BE or having BE without dysplasia. That is, the miRNA are compared to like miRNA. The screening may include: determining an expression level of one or more of miR-15b, miR-21, miR-203, miR-486-5p, and miR-let-7a in the esophageal tissue sample, and comparing the expression level of one or more of miR-15b, miR-21, miR-203, miR-486-5p, and miR-let-7a in the esophageal tissue sample to an expression level of one or more of miR-15b, miR-21, miR-203, miR-486-5p, and miR-let-7a in a tissue sample not associated with BE. Also, the screening may include: providing one or more primers configured to anneal to one or more miR-15b, miR-21, miR-203, miR-486-5p, and miR-let-7a; amplifying one or more of miR-15b, miR-21, miR-203, miR-486-5p, and miR-let-7a, if present in the esophageal tissue sample, using a polymerase; and comparing an amount of one or more of miR-15b, miR-21, miR-203, miR-486-5p, and miR-let-7a in the esophageal tissue sample to an amount of one or more of miR-15b, miR-21, miR-203, miR-486-5p, and miR-let-7a in a tissue sample not having BE or having BE without dysplasia. The primers can be 5′ primers and/or 3′ primers. This method may also be modified in that the detection of BE also detects the presence of BE dysplasia and/or esophageal cancer.

In one embodiment, a method for detecting dysplasia in BE patients can include: collecting an esophageal tissue sample; and screening the esophageal tissue sample for expression of one or more miRNA selected from the group of miR-15b, miR-21, miR-203, miR-486-5p, and miR-let-7a. Such detection of expression can be modulated expression from a standard or control. For example, the standard or control can include comparison to levels of one or more of miR-15b, miR-21, miR-203, miR-486-5p, and miR-let-7a in subjects not having BE or having BE without dysplasia or esophageal cancer. That is, the miRNA are compared to like miRNA. The screening may include: determining an expression level of one or more of miR-15b, miR-21, miR-203, miR-486-5p, and miR-let-7a in the esophageal tissue sample, and comparing the expression level of one or more of miR-15b, miR-21, miR-203, miR-486-5p, and miR-let-7a in the esophageal tissue sample to an expression level of one or more of miR-15b, miR-21, miR-203, miR-486-5p, and miR-let-7a in a tissue sample not having BE or having BE without dysplasia or esophageal cancer. A change in expression level compared to samples not having BE or having BE without dysplasia provides an indication that the sample can include dysplasia or esophageal cancer. Also, the screening may include: providing one or more primers configured to anneal to one or more of miR-15b, miR-21, miR-203, miR-486-5p, and miR-let-7a; amplifying one or more of miR-15b, miR-21, miR-203, miR-486-5p, and miR-let-7a, if present in the esophageal tissue sample, using a polymerase; and comparing an amount of one or more of miR-15b, miR-21, miR-203, miR-486-5p, and miR-let-7a in the esophageal tissue sample to an amount of one or more of miR-15b, miR-21, miR-203, miR-486-5p, and miR-let-7a in a tissue sample not having BE or having BE without dysplasia or esophageal cancer. The primers can be 5′ primers and/or 3′ primers. Here, modulation of amount of miRNA provides an indication the sample has dysplasia or esophageal cancer.

In one embodiment, a method for detecting esophageal cancer, such as EAC, in BE patients can include: collecting an esophageal tissue sample; and screening the esophageal tissue sample for expression of one or more miRNA selected from the group consisting of miR-15b, miR-21, miR-203, miR-486-5p, and miR-let-7a. Such detection of expression can be modulated expression from a standard or control. For example, the standard or control can include comparison to levels of one or more of miR-15b, miR-21, miR-203, miR-486-5p, and miR-let-7a in subjects not having BE or having BE without dysplasia or esophageal cancer. That is, the miRNA are compared to like miRNA. The screening may include: determining an expression level of one or more of miR-15b, miR-21, miR-203, miR-486-5p, and miR-let-7a in the esophageal tissue sample, and comparing the expression level of one or more of miR-15b, miR-21, miR-203, miR-486-5p, and miR-let-7a in the esophageal tissue sample to an expression level of one or more of miR-15b, miR-21, miR-203, miR-486-5p, and miR-let-7a in a tissue sample not having BE or having BE without dysplasia or esophageal cancer. A change in expression level compared to samples not having BE or having BE without dysplasia provides an indication that the sample can include esophageal cancer. Also, the screening may include: providing one or more primers configured to anneal to one or more of miR-15b, miR-21, miR-203, miR-486-5p, and miR-let-7a; amplifying one or more of miR-15b, miR-21, miR-203, miR-486-5p, and miR-let-7a, if present in the esophageal tissue sample, using a polymerase; and comparing an amount of one or more of miR-15b, miR-21, miR-203, miR-486-5p, and miR-let-7a in the esophageal tissue sample to an amount of one or more of miR-15b, miR-21, miR-203, miR-486-5p, and miR-let-7a in a tissue sample not having BE or having BE without dysplasia or esophageal cancer. The primers can be 5′ primers and/or 3′ primers. Here, modulation of amount of miRNA provides an indication the sample has esophageal cancer.

In one embodiment, a method for detecting BE can include: collecting an esophageal tissue sample; and screening the esophageal tissue sample for expression of or change in expression of miRNA selected from miR-15b, miR-21, miR-203, miR-486-5p, and miR-let-7a. Such detection of expression can be modulated expression from a standard or control. For example, the standard or control can include comparison to levels of miRNA selected from miR-15b, miR-21, miR-203, miR-486-5p, and miR-let-7a in subjects not having BE or having BE without dysplasia. That is, the miRNA are compared to like miRNA. The screening may include: determining an expression level of one or more of miR-15b, miR-21, miR-203, miR-486-5p, and miR-let-7a in the esophageal tissue sample, and comparing the expression level of one or more of miR-15b, miR-21, miR-203, miR-486-5p, and miR-let-7a in the esophageal tissue sample to an expression level of one or more of miR-15b, miR-21, miR-203, miR-486-5p, and miR-let-7a in a tissue sample not associated with BE. Also, the screening may include: providing one or more primers configured to anneal to one or more miR-15b, miR-21, miR-203, miR-486-5p, and miR-let-7a; amplifying one or more of miR-15b, miR-21, miR-203, miR-486-5p, and miR-let-7a, if present in the esophageal tissue sample, using a polymerase; and comparing an amount of one or more of miR-15b, miR-21, miR-203, miR-486-5p, and miR-let-7a in the esophageal tissue sample to an amount of one or more of miR-15b, miR-21, miR-203, miR-486-5p, and miR-let-7a in a tissue sample not having BE or having BE without dysplasia. This method may also be modified in that the detection of BE also detects the presence of BE dysplasia and/or esophageal cancer.

In one embodiment, a method for detecting dysplasia in BE patients can include: collecting an esophageal tissue sample; and screening the esophageal tissue sample for expression of one or more miRNA selected from the group of miR-15b, miR-21, miR-203, miR-486-5p, and miR-let-7a. Such detection of expression can be modulated expression from a standard or control. For example, the standard or control can include comparison to levels of one or more of miR-15b, miR-21, miR-203, miR-486-5p, and miR-let-7a in subjects not having BE or having BE without dysplasia or esophageal cancer. That is, the miRNA are compared to like miRNA. The screening may include: determining an expression level of one or more of miR-15b, miR-21, miR-203, miR-486-5p, and miR-let-7a in the esophageal tissue sample, and comparing the expression level of one or more of miR-15b, miR-21, miR-203, miR-486-5p, and miR-let-7a in the esophageal tissue sample to an expression level of one or more of miR-15b, miR-21, miR-203, miR-486-5p, and miR-let-7a in a tissue sample not having BE or having BE without dysplasia or esophageal cancer. A change in expression level compared to samples not having BE or having BE without dysplasia provides an indication that the sample can include dysplasia or esophageal cancer. Also, the screening may include: providing one or more primers configured to anneal to one or more of miR-15b, miR-21, miR-203, miR-486-5p, and miR-let-7a; amplifying one or more of miR-15b, miR-21, miR-203, miR-486-5p, and miR-let-7a, if present in the esophageal tissue sample, using a polymerase; and comparing an amount of one or more of miR-15b, miR-21, miR-203, miR-486-5p, and miR-let-7a in the esophageal tissue sample to an amount of one or more of miR-15b, miR-21, miR-203, miR-486-5p, and miR-let-7a in a tissue sample not having BE or having BE without dysplasia or esophageal cancer. Here, modulation of amount of miRNA provides an indication the sample has dysplasia or esophageal cancer.

In one embodiment, a method for detecting esophageal cancer, such as EAC, in BE patients can include: collecting an esophageal tissue sample; and screening the esophageal tissue sample for expression of one or more miRNA selected from the group consisting of miR-21, miR-203, and miR-let-7a. Such detection of expression can be modulated expression from a standard or control. For example, the standard or control can include comparison to levels of one or more of miR-21, miR-203, and miR-let-7a in subjects not having BE or having BE without dysplasia or esophageal cancer. That is, the miRNA are compared to like miRNA. The screening may include: determining an expression level of one or more of miR-21, miR-203, and miR-let-7a in the esophageal tissue sample, and comparing the expression level of one or more of miR-21, miR-203, and miR-let-7a in the esophageal tissue sample to an expression level of one or more of miR-21, miR-203, and miR-let-7a in a tissue sample not having BE or having BE without dysplasia or esophageal cancer. A change in expression level compared to samples not having BE or having BE without dysplasia provides an indication that the sample can include esophageal cancer. Also, the screening may include: providing one or more primers configured to anneal to one or more of miR-21, miR-203, and miR-let-7a; amplifying one or more of miR-21, miR-203, and miR-let-7a, if present in the esophageal tissue sample, using a polymerase; and comparing an amount of one or more of miR-21, miR-203, and miR-let-7a in the esophageal tissue sample to an amount of one or more of miR-21, miR-203, and miR-let-7a in a tissue sample not having BE or having BE without dysplasia or esophageal cancer. Here, modulation of amount of miRNA provides an indication the sample has esophageal cancer.

In one embodiment, the miRNAs used in the methods described herein can have sensitivity and/or specificity as described herein. The sensitivity and specificity of miRNAs for HGD/EAC were: miR-15b 87% (sensitivity) and 80% (specificity), miR-21 93% (sensitivity) and 70% (specificity), miR-203 87% (sensitivity) and 90% (specificity), miR-486-5p 82% (sensitivity) and 55% (specificity), and miR-let-7a 88% (sensitivity) and 70% (specificity). In one aspect, the sensitivity can be +/−1%, 3%, 5%, 10%, or 15% of the value recited herein. In one aspect, the specificity can be +/−1%, 3%, 5%, 10%, or 15% of the value recited herein.

In one embodiment, the methods described herein can include obtaining healthy esophageal tissue; and comparing miRNA from the healthy esophageal tissue to the miRNA of the test patient. The miRNA from the healthy esophageal tissue may also be compared to miRNA from tissue from BE patients without dysplasia. The miRNA from healthy esophageal tissue may also be compared to miRNA from tissue from BE patients with tissue dysplasia. The miRNA from healthy esophageal tissue may also be compared to miRNA from tissue from BE patients with esophageal cancer. Any of these comparisons can be used to generate standards and controls for comparison with the miRNA of test subjects undergoing the diagnostic procedures.

In one embodiment, the methods described herein can include using a plurality of miRNA biomarkers selected from miR-15b, miR-21, miR-203, miR-486-5p, and miR-let-7a. The methods can include using one or more of miR-15b and miR-486-5p and using one or more of miR-21, miR-203, and miR-let-7a. In one aspect, the methods can include using both miR-15b and miR-486-5p and one or more of miR-21, miR-203, and miR-let-7a. In one aspect, the methods can include using all of miR-15b, miR-21, miR-203, miR-486-5p, and miR-let-7a. In one aspect, the methods can be devoid of using one or more of miR-21, miR-203, and miR-let-7a. In one aspect, the methods can be devoid of using all of miR-21, miR-203, and miR-let-7a. Additionally, the diagnostic kits can include the specific miRNA described in each aspect herein. Reference to such diagnostic kits includes a kit having the miRNA and/or primers thereof, as well as proper reagents for performing the analysis described herein. The primers can be 5′ primers and/or 3′ primers. Additionally, a diagnostic kit or method can include microarrays or other biological chips having primers for or complementary sequences of the miRNA of the present invention.

The sequences for the miRNA biomarkers of the kits and procedures can include: hsa-miR-15b being UAGCAGCACAUCAUGGUUUACA (SEQ ID NO: 1); hsa-miR-486-5p being UCCUGUACUGAGCUGCCCCGAG (SEQ ID NO: 2); hsa-miR-21 being UAGCUUAUCAGACUGAUGUUGA (SEQ ID NO: 3); hsa-miR-203 being GUGAAAUGUUUAGGACCACUAG (SEQ ID NO: 4); hsa-let-7a being UGAGGUAGUAGGUUGUAUAGUU (SEQ ID NO: 5); hsa-let-7d being AGAGGUAGUAGGUUGCAUAGUU (SEQ ID NO: 6); hsa-miR-192 being CUGACCUAUGAAUUGACAGCC (SEQ ID NO: 7); hsa-miR-205 being UCCUUCAUUCCACCGGAGUCUG (SEQ ID NO: 8) and hsa-miR-584 UUAUGGUUUGCCUGGGACUGAG (SEQ ID NO: 9); hsa-miR-375 being UUUGUUCGUUCGGCUCGCGUGA (SEQ ID NO: 10); hsa-miR-1246 being AAUGGAUUUUUGGAGCAGG (SEQ ID NO: 11); hsa-let-7a being UGAGGUAGUAGGUUGUAUAGUU (SEQ ID NO: 12) or complements thereof or primers thereof.

### EXPERIMENTAL

Generally, paired fresh frozen and H&E specimens from a prospective tissue repository included the lesion of interest [i.e., intestinal metaplasia (IM) or high-grade dysplasia (HGD)/esophageal adenocarcinoma (EAC)] occupying >50% of biopsy area. Tissue miRNA expression was determined by microarrays and validated by qRT-PCR. Three groups were compared: Group A includes IM tissues from BE patients without dysplasia; Group B includes IM tissues from Group C patients; and Group C includes dysplastic tissues from BE patients with HGD/EAC.

Barrett's esophageal (BE) and associated neoplastic tissue biopsies used in this study were selected from an ongoing tissue and serum repository of patients with gastroesophageal reflux disease, BE and BE associated dysplasia (Clinical Trials.gov #NCT00574327). The Institutional Review Board of the Veterans Affairs Medical Center, Kansas City, Mo., approved this repository. Patients who presented to the endoscopy unit for evaluation of reflux symptoms or screening/surveillance of BE were invited to participate in the study. All patients signed informed consent and underwent a complete history and physical examination followed by upper endoscopy. BE was defined as presence of columnar lined esophagus on endoscopy with demonstration of intestinal metaplasia in biopsies. Besides clinical care, biopsies were obtained as part of a standardized protocol for collection of specimens for the tissue repository. Per protocol, 2 biopsies were obtained every 2 cm of the BE segment. Immediately, after procurement each 2-3 mm biopsy specimen was divided in half and one half was randomly selected to be fixed in 10% formalin for histopathological evaluation while the other half was placed in RNAlater (Applied Biosystems, Foster City, Calif.) for subsequent RNA extraction. Patients with one or more of the following criteria were excluded from the repository: inability to provide written informed consent, advanced chronic liver disease, severe uncontrolled coagulopathy, and prior history of esophageal or gastric surgery.

Specimens with 4 μm thickness sections were H&E stained and reviewed by a single expert gastrointestinal pathologist. Specimens were examined for the presence of intestinal metaplasia (IM) characterized by the presence of histologically typical goblet cells. The presence of high-grade dysplasia/adenocarcinoma (HGD/EAC) was assessed according to the revised Vienna classification. Only those specimens that exhibited >50% IM or HGD/EAC within the biopsy area on paired H&E sections were used in subsequent RNA analyses. Low-grade dysplasia was excluded in view of variable natural history and poor inter-observer agreement.

Total RNA was extracted using Trizol as per manufacturer's protocol (Sigma, St. Louis, Mo.). Total RNA was quantified using a NanoDrop-1000 spectrophotometer (NanoDrop Technologies, Wilmington, Del.), and quality was assessed by analysis of the 18S and 28S peaks on an Agilent 2001 Bioanalyzer (Agilent Technologies, Santa Clara, Calif.) and calculation of the RNA integrity number (RIN, range 1-10). Only samples with RIN >8 were used for high-throughput microarrays, while the mean and SEM RIN value for all (n=27) samples was 6.2±0.3.

RNA was hybridized individually onto individual Atactic μParaFlo microfluidics chip (Sanger version 12.0, LC Sciences, Houston, Tex.) as described in detail by the manufacturer. These chips included 856 experimentally validated miRNAs. Raw microarray data were preprocessed and normalized according to LC Sciences standard data analysis pipeline (LC Sciences, Houston, Tex.). The raw array data will be made available publically as per the MIAME guidelines. LC Sciences data analysis included the determination of detectable signals, calculation of signal intensities, and calculation of differential ratios. First, background subtraction, Cy3/Cy5 channel normalization, detectability determination, and p-value calculation were performed. The background was determined using a regression-based background mapping method. The regression was performed on 5% to 25% of the lowest intensity data points excluding blank spots and the background matrix was then subtracted from the raw data matrix. Normalization was carried out using a locally-weighted regression (LOWESS) method on the background-subtracted data. A transcript was listed as detectable if it met at least two conditions: signal intensity higher than 3*(background standard deviation) and (spot CV)<0.5, where (spot CV) is calculated by (standard deviation)/(signal intensity). Multiple sample analysis normalization was performed using a cyclic LOWESS method.

To evaluate ranking among miRNAs, a basic linear regression model was built based on mean miRNA signal intensities between the BE groups with and without dysplasia. First, we calculated mean signal intensities within each group for each miRNA. Next, we built a linear model between mean signal intensities. The linear model gave us the general relationship of an individual miRNA's mean intensity values between the two groups. In this model, the miRNAs that were not differentially expressed followed the linear model and those that were significantly differentially expressed deviated from the model. Therefore, we could rank the miRNAs according to their deviation from the linear model that allowed the selection of differentially expressed miRNAs for the final validation step. Here, we used the distance from the regression line as a measure of deviation from the linear model. MicroRNAs farther from the regression line were assigned higher rank than those closer to the line.

Candidate miRNA biomarkers from the results of the above model were tested further in an independent validation group of BE patients with and without dysplasia. The miRNA assayed included: hsa-miR-1246 with 2.11 fold change; hsa-miR-145 with 0.35 fold change; hsa-miR-15b with 4.18 fold change; hsa-miR-143 with 0.92 fold change; hsa-miR-192 with 2.93 fold change; hsa-miR-196a with 2.76 fold change; hsa-miR-215 with 0.7 fold change; hsa-miR-203 with 11.76 fold change; hsa-miR-205 with 6.53 fold change; hsa-miR-21 with 12.57 fold change; hsa-miR-let-7d with 0.7 fold change; hsa-miR-let-7c with 1.96 fold change; hsa-miR-let-7b with 0.84 fold change; hsa-miR-let-7a with 3.06 fold change; hsa-miR-375 with 1.25 fold change; hsa-miR-486-5p with 0.15 fold change; and hsa-miR-584 with 4.43 fold change; and hsa-miR-106b with 2.5 fold change. Candidate miRNA were chosen based on the following criteria: i) the log of fold change of a miRNA should have been either greater than 1 or less than −1 (indicating 2-fold up or down regulation), ii) the miRNA of interest should have a multiple testing adjusted P<0.05 from the moderate t-test using R package LIMMA (21). Additional miRNAs (miR-145, miR-196a, miR-203, and miR-375) were tested.

Quantitative RT-PCR (qRT-PCR) was performed to validate microarray results using procedures explained in detail previously by authors (22, 23). Briefly, 250 ng of total RNA was used in the Qiagen (Valencia, Calif.) or 25 ng was used in the Exiqon (Woburn, Mass.) reverse transcription reactions following the manufacturer's protocol. Primers for miR-15b, -21, -145, -192, -203, -205, -375, and -1246 were designed in house or purchased (Qiagen) and detected using the miScript PCR system (Qiagen). Primers for miR-196a, miR-486-5p, miR-584, miR-let-7a and miR-let-7d were purchased from Exiqon and detected using the Universal cDNA Synthesis and SYBR Green kits (Exiqon). The miRNA primers for U6, the small nuclear U6 RNA, were obtained from Qiagen or Exiqon and used to normalize for starting material. The qRT-PCR reactions were completed on the 7900 HT Sequence Detection System (Applied Biosystems). Each primer set included a minus RT control. All patient samples were run in triplicate, and the average used in subsequent calculations. Briefly, the delta-delta Ct method was used to calculate relative fold change values between patient samples and for calculation of means and s.e.m. values. All samples being compared to a single patient in order to calculate fold changes.

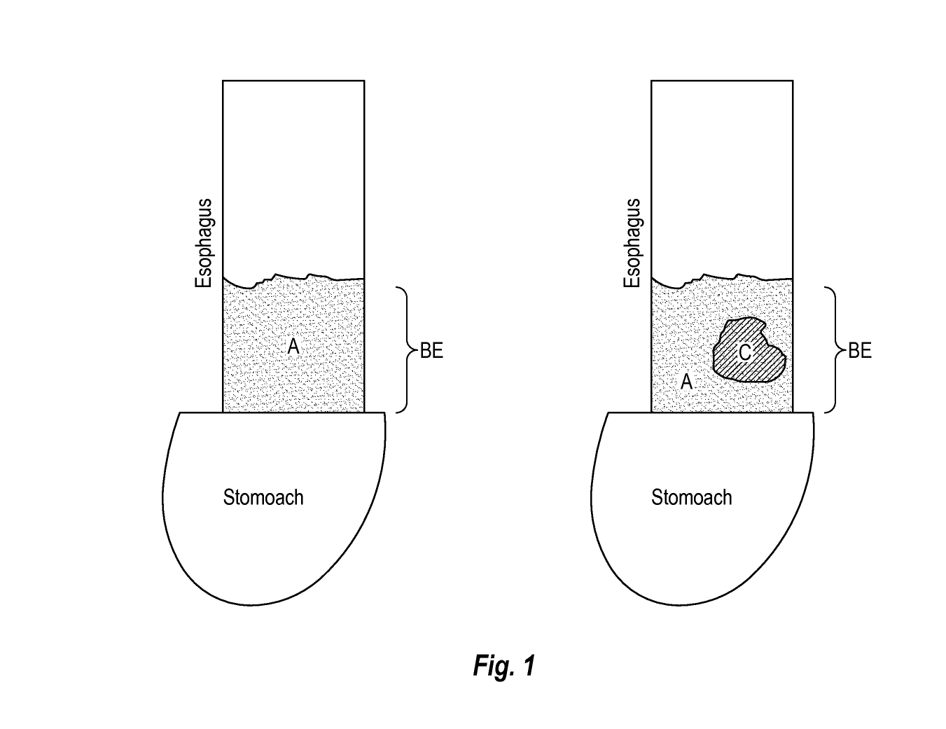

The three main comparison groups (FIG. 1) comprised of: Group A, intestinal metaplasia tissues from BE patients without dysplasia; Group B, intestinal metaplasia tissues from BE patients with high grade dysplasia/esophageal adenocarcinoma (BE-HGD/EAC) patients (i.e., Group C patients); and Group C, dysplastic tissues from BE-HGD/EAC patients.

Quantitative RT-PCR results were log transformed if positive Bartlett's tests for unequal variances were determined. The qRT-PCR results (fold changes) were then subjected to ANOVA across the three groups. Upon detection of a significant F-test by ANOVA, Tukey's Multiple Comparison Tests (i.e., post-hoc mean separation tests) were completed to determine which groups differed. Receiver operator curves (ROC) using fold change were constructed for the differentially expressed miRNAs between the various groups. A P value <0.05 was considered significant.

The data set was obtained from 22 BE patients, were 11 patients had dysplasia and 11 patients dot not have dysplasia, where the BE patients had a mean age 64±8.2 years and 63±11.6 years respectively. All patients were Caucasian males. Nine miRNAs were identified by high-throughout analysis: miR-15b, miR-21, miR-192, miR-205, miR-486-5p, miR-584, miR-1246, miR-let-7a and miR-7d). Of these, qRT-PCR confirmed expression of miR-15b, -21, 486-5p and let-7a. Two miRNAs, miR-145 and miR-203 exhibited differential expression. The miR-196a and miR-375 miRNAs did not exhibit differential expression.

Sensitivity and specificity of miRNAs for HGD/EAC were: miR-15b 87% (sensitivity) and 80% (specificity), miR-21 93% (sensitivity) and 70% (specificity), miR-203 87% (sensitivity) and 90% (specificity), miR-486-5p 82% (sensitivity) and 55% (specificity), and miR-let-7a 88% (sensitivity) and 70% (specificity).

Additionally, miR-15b, miR-21 and miR-203 exhibited field effects, which indicated that Group A and Group B tissues while being histologically similar, yet exhibited different miRNA expression.

Mean BE length was C3M4.3 (range COM2-C11M14) cm in the BE patients without dysplasia and C4.3M5.1 (COM3-C7M8) in those with dysplasia. All patients demonstrated hiatus hernia with mean length of 3.5 cm in each group. Fifty percent of the BE patients without dysplasia were active smokers compared to 30% of the dysplastic patients (P=NS). All patients were receiving anti-secretory therapy for at least 6 months at the time of the procedure and none of the patients had erosive esophagitis. For the final analysis, these 22 patients contributed 27 biopsy specimens (Group A: 11; Group B: 7 and Group C: 9). Of the 11 patients included in groups B & C, 5 contributed to both groups, 2 only to group B and 4 only to group C.

Histological analysis of each biopsy specimen was used to calculate the percent of lesion that occupied the biopsy area. The mean biopsy surface area occupied by intestinal metaplasia in Group A was 59% (range 51-100%) and Group B was 63% (range 50%-100%), P=NS. The mean biopsy surface area occupied by HGD/EAC in the biopsies from Group C (BE patients with HGD/EAC) patients was 67% (range 50%-100%). The mean distance of the non-dysplastic biopsies (Group B) from the dysplastic biopsies (Group C) was 3.2 cm. In Group A and Group B biopsies, the histology other than the lesion of interest was columnar mucosa (cardiac/fundic type tissue), but without the specialized intestinal metaplasia/goblet cells. For Group C biopsies, the remaining histology was also columnar mucosa (cardiac/fundic/specialized intestinal metaplasia with goblet cells). None of the specimens had non-columnar, squamous mucosa.

Analysis of the microarray results from tissue biopsies from 4 patients (2 with non-dysplastic intestinal metaplasia and 2 with high-grade dysplasia) identified nine miRNAs that were differentially expressed between the two groups. The majority (8 of 9) of these differentially expressed miRNA exhibited increased expression: miR-15b, miR-21, miR-192, miR-205, miR-584, miR-1246, let-7a, and let-7d. On the other hand, only one miR-486-5p had reduced expression in patients with HGD/EAC.

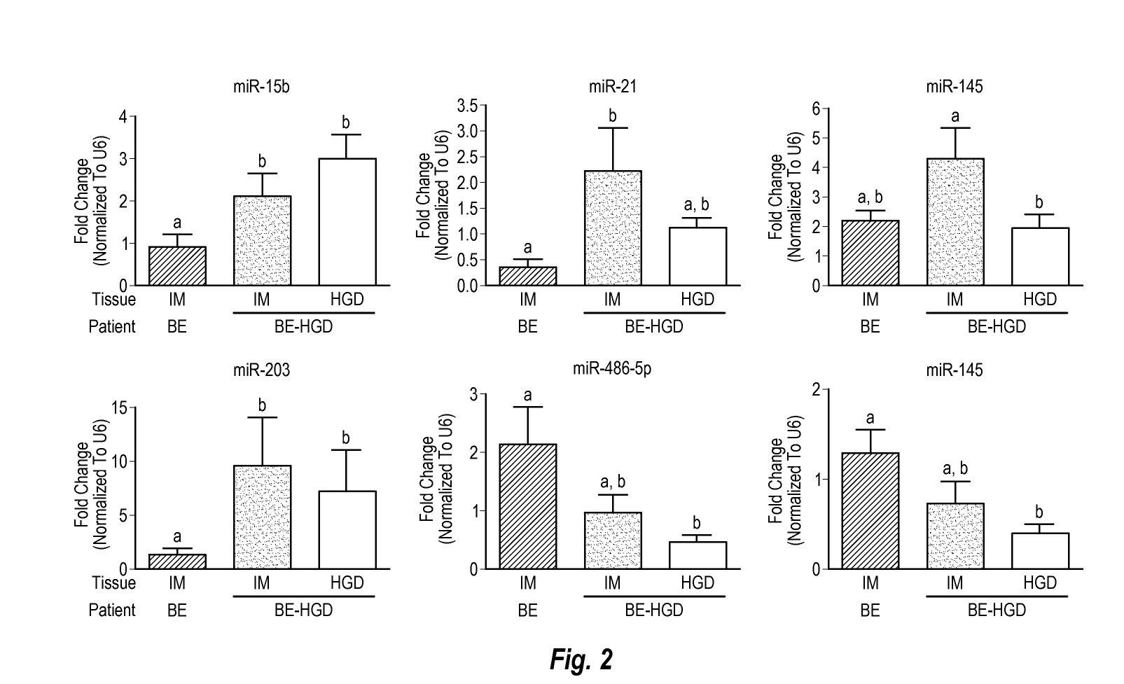

FIG. 2 depicts data for miRNA that were differentially expressed following qRT-PCR validation in the larger patient cohorts for three tissue groups. Group A (e.g., non-dysplastic intestinal metaplasia) was compared with Group C (e.g., HGD/EAC), and quantitative RT-PCR analysis confirmed differential expression for 4 miRNA: miR-15b (3.3-fold), miR-203 (5.7-fold), miR-486-5p (−4.8 fold) and let-7a (−3.3 fold), where a “+” indicates an increase in Group C and a “−” indicates decrease in Group C. The miR-15b (2.4-fold) and miR-203 (7.6-fold) biomarkers were also greater (P<0.05) in Group B than Group A, supporting presence of a field effect in miRNA expression. It should be noted that Group A and Group B tissues both are histologically non-dysplastic intestinal metaplasia. MicroRNA-21 expression was also greater (P<0.05, 6.6-fold) in Group B versus Group A tissues (FIG. 2). MicroRNA-145 expression was greater (P<0.05, 2.2-fold) in Group B tissues than Group C tissues. The remaining miRNA, miR-192, miR-196a, miR-205, miR-375, miR-584, miR-1246 and miR-let-7d, were not validated in the qRT-PCR analysis of the larger patient cohorts; however, these may still be used in the diagnostic kits and assays as described herein.

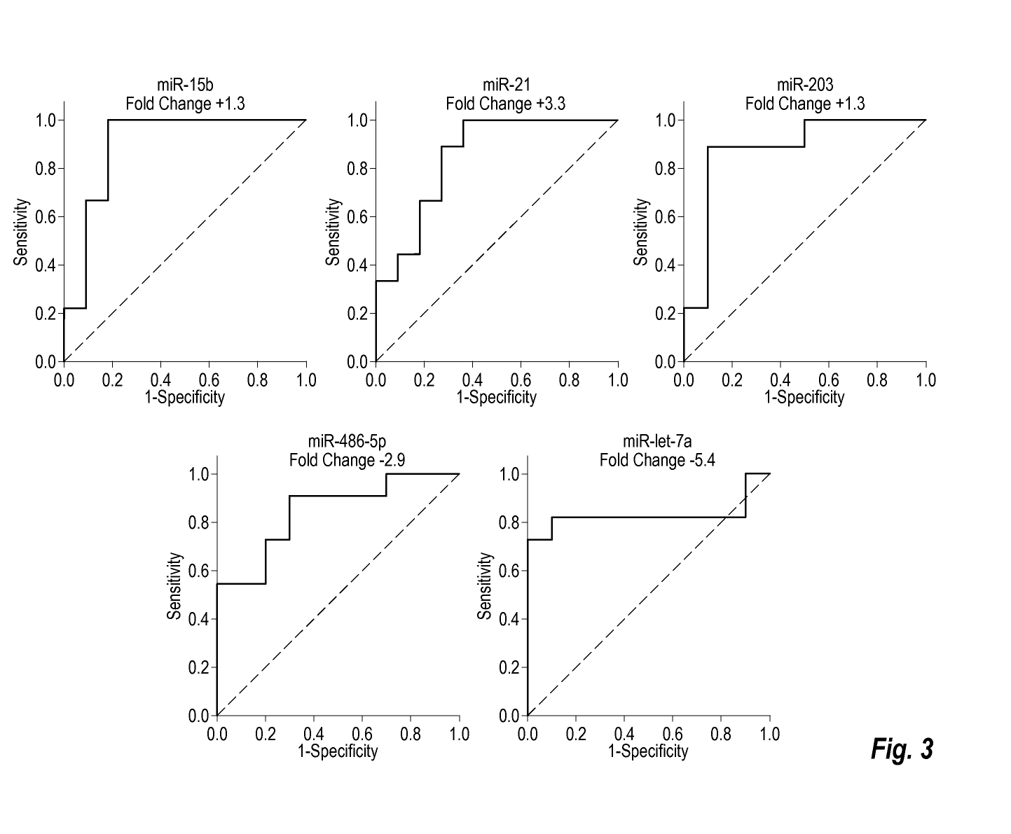

The receiver operator characteristics of miR-15b, miR-21, miR-203, miR-486-5p and let-7a were assayed for prediction of patients being susceptible to or progressing to dysplasia. To quantify the feasibility of miRNAs to discriminate patients with and without dysplasia, we constructed ROC curves for 5 miRNA. FIG. 3 depicts the sensitivity and specificity for these 5 miRNA that compare BE patients with no dysplasia (Group A) with BE-HGD/EAC (Group C only) as follows: miR-15b 100% and 80% (AUC 0.889, P=0.004), miR-21 80% and 67% (AUC 0.833, P=0.014), miR-203 89% and 90% (AUC 0.878, P=0.006), miR-486-5p 91% and 70% (AUC 0.845, P=0.007) and miR-let-7a 82% and 90% (AUC 0.827, P=0.011).

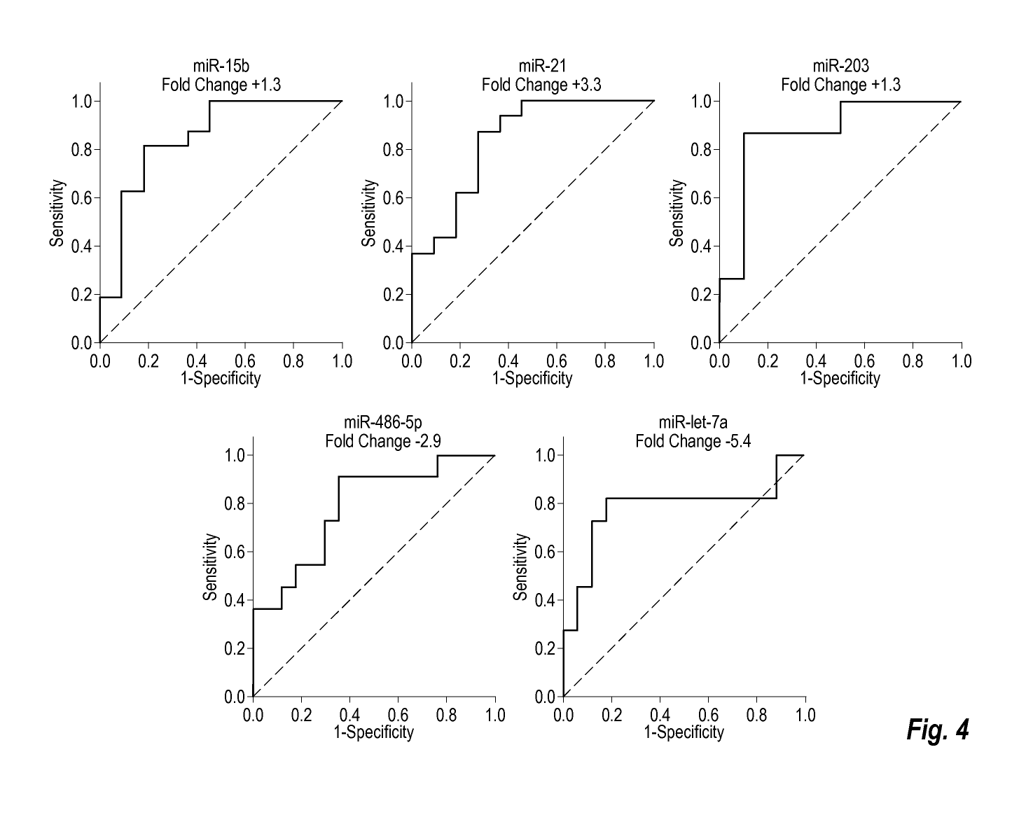

To discriminate the BE patients with the overall diagnosis of dysplasia from those without dysplasia by using global miRNA expression, a diagnosis of BE with non-dysplastic intestinal metaplasia (Group A) was compared to non-dysplastic and dysplastic tissue biopsies from BE-HGD/EAC patients (Groups B and C combined). FIG. 4 shows the sensitivity and specificity of 5 miRNAs: miR-15b 87% and 80% (AUC 0.847, P=0.003), miR-21 93% and 70% (AUC 0.841, P=0.003), miR-203 87% and 90% (AUC 0.873, P=0.002), miR-486-5p 82% and 55% (AUC 0.845, P=0.007) and miR-let-7a 88% and 70% (AUC 0.827, P=0.011).

Additionally, the findings in FIG. 4 are particularly relevant and can be useful in the kits and procedures described herein. The findings can improve the clinical applicability of this invention. These findings are useful in utilizing the concept of “field effect,” which means that in a BE patient with high-grade dysplasia/cancer, the BE lining is abnormal everywhere and not just the location of the endoscopically apparent dysplasia/cancer. The field effect can refer to the effect of the abnormal tissue having an effect on other tissue in a field there-around. Also, “field effect” may also be referred to as a “field defect” due to the abnormal defect being delocalized and in a field around a certain tissue area. As such, the tissue obtained for the present invention can be obtained via a cytology brush. The cytology brush can be used to obtain cells and tissue samples from a wide area in the esophagus, and these cells and tissues can be used to obtain test miRNA. The cytology brush allows for cells and tissues to be obtained for the diagnostic procedures by manually insertion into the esophagus without specific tissue dissection. The diagnostic procedure allows the cytology brush to be blindly passed without an endoscopy in a subject's esophagus in the clinical office of a gastroenterologist. Then, the cytologically obtained cells can be evaluated for the miRNA, such as miR-15b, miR-21, miR-203, miR-486-5p, and miR-let-7a in order to predict susceptibility to or diagnose dysplasia or cancer. Thus, the field effect and cytology brush can extend the reach of the diagnostic procedures to diagnose esophageal adenocarcinoma. For example, the field effect and cytology brush can be used to obtain samples from locations where endoscopy is not readily available. Thus, the diagnostic kits and procedures can include a cytology brush having a length sufficient to extend from the mouth to esophagus of a subject.

In view of the foregoing, the data show that the miRNA of the diagnostic kits and procedures described herein can be used to differentiate BE patients with and without dysplasia with reasonable clinical accuracy.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims. All references recited herein are incorporated herein by specific reference in their entirety.

1. A method of diagnosing progression from Barrett's esophagus toward esophageal dysplasia, the method comprising:
obtaining miRNA from a test subject having Barrett's esophagus;
assaying for miRNA biomarker selected from one or more of miR-15b or miR-486-5p; and
determining whether one or more of miR-15b or miR-486-5p provide an indication of progression toward esophageal dysplasia in the test subject.

2. The method of claim 1, comprising determining an amount of one or more of miR-15b or miR-486-5p.

3. The method of claim 1, comprising determining a modulation in amount of one or more of miR-15b or miR-486-5p.

4. The method of claim 1, comprising comparing the one or more of miR-15b or miR-486-5p with a positive control or a negative control, the positive control being from a subject having esophageal dysplasia, the negative control being from a subject without esophageal dysplasia.

5. The method of claim 4, wherein if one or more of the miR-15b or miR-486-5p is similar to the positive control or different from the negative control, there is an indication of esophageal dysplasia in the test subject.

6. The method of claim 4, wherein if one or more of the miR-15b or miR-486-5p is similar to the negative control or different from the positive control, there is an indication of esophageal dysplasia in the test subject.

7. The method of claim 1, comprising:
assaying for miRNA biomarker selected from one or more of miR-21, miR-145, miR-203, or miR-let-7a; and
determining whether one or more of miR-21, miR-145, miR-203, or miR-let-7a provide an indication of esophageal dysplasia in the test subject.

8. The method of claim 1, wherein the assaying includes contacting the miRNA biomarker with a complement thereof.

9. The method of claim 8, comprising performing RT-PCR with one or more primers for the miRNA biomarker.

10. The method of claim 8, comprising the complement of the miRNA biomarker being located on a diagnostic chip.

11. The method of claim 1, comprising:
diagnosing esophageal dysplasia in the test subject.

12. The method of claim 1, comprising:
diagnosing esophageal cancer in the test subject.

13. A method of detecting progression from Barrett's esophagus toward dysplasia, the method comprising:
obtaining miRNA from a test subject;
assaying for a plurality of miRNA biomarkers selected from the group consisting of miR-15b, miR-486-5p, miR-21, miR-145, miR-203, or miR-let-7a; and
determining whether the assayed plurality of miRNA selected from the group consisting of miR-15b, miR-486-5p, miR-21, miR-145, miR-203, or miR-let-7a provide an indication of progression toward dysplasia in the test subject.

14. The method of claim 13, comprising determining an amount or modulation of the assayed plurality of miRNA selected from the group consisting of miR-15b, miR-486-5p, miR-21, miR-145, miR-203, or miR-let-7a.

15. The method of claim 13, comprising:
comparing the assayed plurality of miRNA selected from the group consisting of miR-15b, miR-486-5p, miR-21, miR-145, miR-203, or miR-let-7a with a positive control or a negative control, the positive control being from a subject having dysplasia, the negative control being from a patient without dysplasia,
wherein if one or more of the miR-15b or miR-486-5p is similar to the positive control or different from the negative control, there is an indication of progression toward dysplasia in the test subject; or
if one or more of the miR-15b or miR-486-5p is similar to the negative control or different from the positive control, there is an indication of progression toward dysplasia in the test subject.

16. The method of claim 13, comprising:
diagnosing esophageal dysplasia in the test subject.

17. The method of claim 13, comprising:
diagnosing esophageal cancer in the test subject.

18. A diagnostic kit comprising:
a plurality of polynucleotides selected from the group consisting of miR-15b, miR-486-5p, miR-21, miR-145, miR-203, miR-let-7a, or compliment thereof.

19. The diagnostic kit of claim 15, comprising one or more of:
miR-15b or complement thereof or miR-486-5p or complement thereof.

20. The diagnostic kit of claim 18, the plurality of nucleotides including one or more primers for two or more of miR-15b, miR-486-5p, miR-21, miR-145, miR-203, or miR-let-7a.

21. The diagnostic kit of claim 18, the plurality of nucleotides including a compliment polynucleotide for two or more of miR-15b, miR-486-5p, miR-21, miR-145, miR-203, or miR-let-7a.

22. The diagnostic kit of claim 21, a chip having the plurality of compliment nucleotides for two or more of miR-15b, miR-486-5p, miR-21, miR-145, miR-203, or miR-let-7a.


SEQUENCE LISTING (12 sequences)

SEQ ID NO: 1 - RNA, 22 nt, Homo sapiens
uagcagcacaucaugguuuaca

SEQ ID NO: 2 - RNA, 22 nt, Homo sapiens
uccuguacugagcugccccgag

SEQ ID NO: 3 - RNA, 22 nt, Homo sapiens
uagcuuaucagacugauguuga

SEQ ID NO: 4 - RNA, 22 nt, Homo sapiens
gugaaauguuuaggaccacuag

SEQ ID NO: 5 - RNA, 22 nt, Homo sapiens
ugagguaguagguuguauaguu

SEQ ID NO: 6 - RNA, 22 nt, Homo sapiens
agagguaguagguugcauaguu

SEQ ID NO: 7 - RNA, 21 nt, Homo sapiens
cugaccuaugaauugacagcc

SEQ ID NO: 8 - RNA, 22 nt, Homo sapiens
uccuucauuccaccggagucug

SEQ ID NO: 9 - RNA, 22 nt, Homo sapiens
uuaugguuugccugggacugag

SEQ ID NO: 10 - RNA, 22 nt, Homo sapiens
uuuguucguucggcucgcguga

SEQ ID NO: 11 - RNA, 19 nt, Homo sapiens
aauggauuuuuggagcagg

SEQ ID NO: 12 - RNA, 22 nt, Homo sapiens
ugagguaguagguuguauaguu